(19) United States
(12) Patent Application Publication
(10) Pub. No.: US 2010/0151035 A1
(43) Pub. Date: Jun. 17, 2010

(54) PHARMACEUTICAL COMPOSITIONS OF POORLY SOLUBLE DRUGS

(75) Inventors: Bharatrajan Ramaswami, Thane (IN); Manisha Rajesh Patil, Thane (IN); Aditi Das, Thane (IN)

Correspondence Address: MERCHANT & GOULD PC, P.O. BOX 2903, MINNEAPOLIS, MN, 55402-0903, US

(73) Assignee: Sandoz AG, Basel (CH)

(21) Appl. No.: 12/529,504

(22) PCT Filed: Mar. 10, 2008
(86) PCT No.: PCT/EP08/52823

§ 371 Date: Sep. 1, 2009

(30) Foreign Application Priority Data
Mar. 13, 2007 (EP) 07103985.3
Oct. 31, 2007 (IN) 2166/MUM/2007

(51) Int. Cl. A61K 9/16; A61K 31/56; A61K 38/43; A61K 38/02; A61K 38/16; A61K 31/165; A61K 31/437; A61K 31/4184; A61K 31/415; A61K 38/13; A61K 31/573; A61K 31/397; A61K 31/235; A61K 31/403; A61K 31/495; A61K 31/343; A61K 31/496; A61K 31/202; A61K 31/5377; A61K 31/436; A61K 31/138; B05D 1/02

(52) U.S. Cl. 424/497; 514/169; 424/94.1; 514/2; 514/12; 514/618; 514/288; 514/381; 514/406; 514/11; 514/180; 514/210.02; 514/543; 514/412; 514/255.06; 514/462; 514/253.01; 514/559; 514/236.8; 514/291; 514/648; 514/395; 427/2.14

(57) ABSTRACT

The present invention relates to a stable pharmaceutical composition of a poorly water-soluble drug with a view to increasing its solubility and bioavailability. The present invention relates to a solid dispersion of a poorly water-soluble drug.

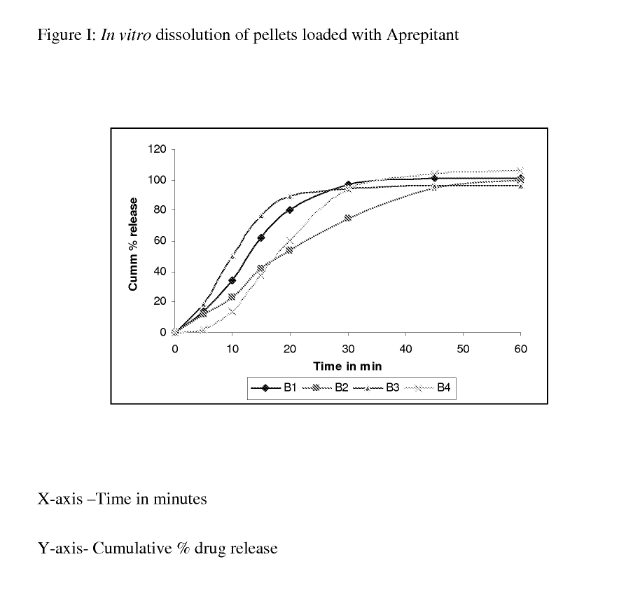

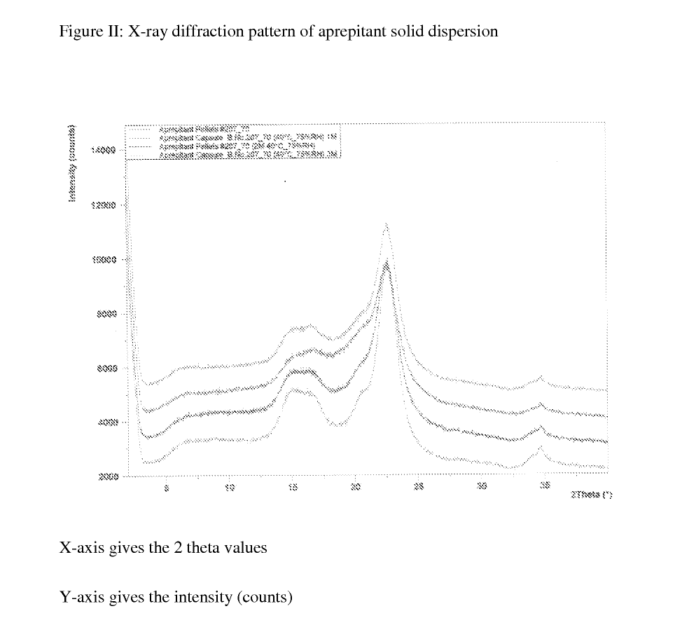

### FIELD OF THE INVENTION

The present invention relates to pharmaceutical compositions of poorly soluble drugs. The present invention relates to solid dispersions of poorly water soluble drugs. The present invention also relates to a process for preparing solid dispersions.

### BACKGROUND OF THE INVENTION

Drugs that are poorly water-soluble are usually characterized by low absorption and poor bioavailability and present special difficulties when formulating dosage form. The bioavailability of many poor water-soluble drugs is limited by the dissolution rate, which in turn is governed by the particle size and hence specific surface area and/or the polymorphic state of the active ingredient.

Bioavailability is defined as the degree to which a drug becomes available to the target tissue after administration. Absorption of the drug in the body is dependent on the bioavailability of the drug. To facilitate absorption, the drug must be in soluble form at the site of absorption.

Various techniques are employed to increase the solubility of the drug which include, but are not limited to, decreasing the particle size, complexation, formation of a solid solution, changing the surface characteristics of the particles and incorporation of drug particles into colloidal systems like nanoparticles and liposomes.

The rate of dissolution of a drug is inversely proportional to the particle size of the drug. Consequently, methods of making finely-divided drug have been studied and efforts have been made to control the size of drug particles in pharmaceutical compositions. Techniques such as dry milling, wet grinding and commercial airjet milling have provided particles with an average particle size ranging from as low as about 1 μm to about 50 μm (1,000-50,000 nm).

Further attempts have been made to improve the bioavailability of poorly water-soluble drugs by various techniques such as lyophilization, solvate formation and solid dispersion. Advantages and techniques to improve the solubility of poorly water-soluble drugs by formation of solid dispersions have been described in the literature (Pharm. Acta Hely. 61(3), 1986, pp. 69-88).

A solid dispersion is a pharmaceutical formulation which may be defined as a dispersion of one or more active ingredient in an inert carrier, or matrix at solid state prepared by melting (fusion), solvent or melting-solvent methods.

The melting method of preparing a solid dispersion includes fusion of the two components where the drug and carrier are allowed to melt at or above the melting point of the drug. The molten mixture is then cooled rapidly to provide a congealed mass, which is subsequently milled to produce a powder. The fusion process is technically simple if the drug and the carrier are miscible in the molten state. This process cannot be used for those drugs which decompose on heating thus has its own limitations.

The melting-solvent method involves dissolution of the drug in a small amount of organic solvent, which is then added to the molten carrier. The solvent is then evaporated to generate a product that is subsequently milled to produce a powder.

The solvent-based process uses organic solvents to dissolve and disperse the drug and carrier molecule. The solvent is later removed by evaporation and the drug-carrier dispersion is collected as a powdered mass.

2-(R)-(1-(R)-(3,5-bis(trifluoromethyl)phenyl)ethoxy)-3-(S)-(4-fluoro)phenyl-4-(3-(5-oxo-1H,4H-1,2,4-triazolo)methylmorpholine(aprepitant) is an antagonist of Substance P, which is a naturally-occurring undecapeptide belonging to the tachykinin family of peptides. There is evidence for the use of a tachykinin receptor antagonist in the treatment of various diseases or disorders. Aprepitant is poorly water soluble and thus exhibits slow oral absorption, resulting in very low oral bioavailability. Thus, there is a need to increase the solubility and the bioavailability of Aprepitant

Various methods for the formulation of poorly soluble drugs are described in the literature.

European Patent 499,299 describes a technique for preparing pharmaceutical compositions comprising loading drugs into liposomes or polymers. Such techniques have problems and limitations. For example, a lipid soluble drug is often required for preparing suitable liposomes. Further, unacceptably large amounts of the liposome or polymer are often required to prepare unit drug doses. Furthermore, techniques for preparing such pharmaceutical compositions tend to be complex. A principal technical difficulty encountered with emulsion polymerization is the removal of contaminants, such as unreacted monomer or initiator, which can be toxic at the end of the manufacturing process.

U.S. Pat. No. 4,540,602 (the '602 patent) discloses a method for the preparation of an activated pharmaceutical composition containing a solid drug. A sparingly soluble drug is dispersed in water in the presence of a water-soluble high-molecular weight substance to form a disperse system containing the drug in the form of finely divided particles substantially not greater than 10 μm in diameter. The dispersion medium is then removed from the disperse system, whereby a pharmaceutical composition containing finely divided drug coated with the water-soluble high-molecular substance is obtained. The '602 patent teaches various methods for dispersing the drug in water to form a disperse system containing the drug in the form of finely divided particles. Pulverization of drug in water is one of the techniques.

European Patent 275,796 (the '796 patent) describes the production of colloidally dispersible systems comprising a substance in the form of spherical particles smaller than 500 nm This method involves a precipitation effected by mixing a solution of the substance and a miscible non-solvent for the substance, and results in the formation of non-crystalline nanoparticles. According to the '796 patent, precipitation techniques for preparing particles tend to provide particles contaminated with solvents. Such solvents are often toxic and it can be very difficult, if not impossible, to reduce the solvent content to pharmaceutically acceptable levels to be practical.

U.S. Pat. No. 5,145,684 describes stable, dispersible drug nanoparticles and a method for preparing such particles by wet milling in the presence of grinding media in conjunction with a surface modifier. The particles can be formulated into pharmaceutical compositions exhibiting remarkably high bioavailability.

U.S. Pat. No. 6,881,745 describes a process for preparing solid dispersion of a poorly water soluble drug. The invention involves dissolution of the polymer which has acidic functional group in a solvent and adding the drug in the solvent to form a suspension which is spray dried to form solid dispersion.

U.S. Pat. No. 5,633,015 describes a bead comprising an inert core, coated with anti-fungal and a hydrophilic polymer and seal coating polymer. The patent describes dosage forms comprising such beads and the process for making them.

U.S. Patent Application 2004/0214746 describes processes involving nanoparticulate technology to increase solubility and improve bioavailability of less soluble drugs such as 2-(R)-(1-(R)-(3,5-bis(trifluoromethyl)phenyl)ethoxy)-3-(S)-(4-fluoro)phenyl-4-(3-(5-oxo-1H,4H-1,2,4-triazolo)methylmorpholine. The process described in this application includes wet grinding as one of the techniques to increase solubility.

PCT application 2007/016582 describes co-precipitates comprising amorphous aprepitant and pharmaceutically acceptable carriers.

The preparation and characterization of solid dispersion on pellets using fluidized bed system is described in International Journal of Pharmaceutics 139, (1996) 223-229.

Most of the prior art techniques described suffer from disadvantages of using solvents or melting the drug and carrier to give pharmaceutical compositions of improved bioavailability. Moreover, prior art methods are multi-step processes for the preparation of solid solutions. Hence, there is a need for a single step process for the preparation of stable compositions of poorly soluble drugs.

It would, therefore, be desirable to provide stable pharmaceutical compositions of poorly soluble drugs which can be readily prepared and that show improved solubility and, in turn, improved bioavailability over the drug substance.

### SUMMARY OF THE INVENTION

The present invention relates to a stable pharmaceutical composition of a poorly soluble drug. The present invention also relates to a process for the preparation of a pharmaceutical composition containing poorly soluble drug comprising the steps of:
a) dissolving the drug, or a pharmaceutically acceptable salt thereof, and at least one polymer in a suitable solvent, to form a solution;
b) spraying the solution onto inert pellets; and
c) drying the inert pellets to remove the solvent.

### BRIEF DESCRIPTION OF THE DRAWINGS

Figure I: Dissolution of pellets loaded with aprepitant.

Figure II: XRD studies on the Aprepitant formulations under at various time points under stress conditions.

### DETAILED DESCRIPTION OF THE INVENTION

The present invention relates to a stable pharmaceutical composition of a poorly soluble drug. The present invention further provides a pharmaceutical composition wherein the dissolution rate of the drug is dependent on the particle size of the inert pellets.

The present invention further relates to a process for the preparation of a stable pharmaceutical composition of a poorly soluble drug. Specifically, the present invention relates to a process for the preparation of a solid dispersion of a poorly water-soluble drug. The present invention relates to a process for the preparation of a pharmaceutical composition comprising a poorly soluble drug, comprising the steps of:
a) dissolving the drug, or a pharmaceutically acceptable salt thereof, and at least one polymer in suitable solvent, to form a solution;
b) spraying the solution onto inert pellets; and
c) drying the inert pellets to remove the solvent.

One embodiment of the present invention relates to a process for the preparation of a stable pharmaceutical composition of aprepitant. One embodiment of the present invention provides a process for the preparation of a solid dispersion of aprepitant comprising the steps of:
a) dissolving aprepitant, or a pharmaceutically acceptable salt thereof, and at least one polymer in a suitable solvent, to form a solution;
b) spraying the solution onto inert pellets; and
c) drying the inert pellets to remove the solvent.

Another embodiment of the present invention provides a process for the preparation of a solid dispersion of aprepitant wherein the dissolution rate of aprepitant is dependent on the particle size of the inert pellets contained in the composition.

Various terms that will be used throughout this specification have meaning that will be well understood by person skilled in the art. For ease of reference, however, some of these terms are defined.

In accordance with the present invention, “solid dispersion” means a solid-state system containing at least two components wherein the first component is dispersed rather uniformly in the second component. The dispersed materials maintain the system in a chemically or physically uniform or homogeneous state, or maintain the system in one phase as defined in thermodynamics The solid solution contains materials in super-homogeneous state, such as glassy solid solution, as well as in less homogeneous state.

The term “poorly water soluble” as used herein applies to drugs that are essentially totally water-insoluble or practically insoluble. Specifically, the term is applied to any drug that has aqueous solubility less than 1.0 mg/ml in unbuffered water.

The term “drug” means a compound having beneficial prophylactic and/or therapeutic properties when administered to humans

The term “organic solvent” as used herein includes, but is not limited to, lower alcohols, chlorinated solvents (1,2-dichloromethane, chloroform) or any other pharmaceutically acceptable solvents and mixtures thereof.

Formation of a solid dispersion does not result in the formation of a covalent bond and the drug does not form a lattice structure which would result in crystal formation. Instead, it results in the formation of a solid solution.

In a solid dispersion, particle size reduction of the drug within the matrix is achieved to the minimum level, i.e., the molecular state is achieved. As the carrier dissolves, the drug present in the molecular form leads to the formation of a supersaturated solution. This leads to enhancement of the dissolution rate of the poorly soluble drug, and results in an increase in bioavailability.

As the solid dispersion is exposed to water or gastro-intestinal juices, the water-soluble carrier is released to the internal aqueous solution. Simultaneously, components of the solid dispersions dissolve into minute particles, which increase the surface area of the drug. At this time, the drug particles become smaller and the carrier dissolves completely in a very short time, so that the solubilization of drug is achieved by the carrier in a diffusion layer, which is a minute environment around the drug particles at the early stage of dissolution. Therefore, it is understood that the above-mentioned factors work collectively to increase the solubility and initial dissolution rate of drug.

Solid dispersion is generally prepared by organic solvent evaporation method since relatively low temperature is required. Thus, the thermal degradation of drugs can be avoided.

A modification of the organic solvent evaporation method involves spraying the drug-carrier solution onto granular surface of excipient or onto a solid support like pellets. This solution upon coating and controlled drying of the coated pellets produces a solid dispersion of the drug on the pellet or solid support. Complete removal of the solvent to trace levels to comply with regulations and a possibility of formation of solvates may limit pharmaceutical acceptance. Due to toxicity issues and unwanted side effects on drug stability, complete removal of solvents to trace level is recommended.

Any water insoluble drug may be formulated in the practice of the present invention so as to increase its solubility and hence its bioavailability. Drugs that are particularly useful in the practice of the present invention include the drug from the class of antibacterial, antacids, analgesic and anti-inflammatory agents, anti-arrhythmic agents, antiprotozoal agents, anti-coagulants, antidepressants, anti-diabetic agents, anti-epileptic agents, antifungal agents, antihistamines, anti-hypertensive agents, anti-muscarnic agents, antineoplastic agents, antimetabolites, anti-migraine agents, anti-Parkinsonian agents, antipsychotic, hypnotic and sedating agents, anti-stroke agents, antitussive, antivirals, cardiac inotropic agents, corticosteroids, disinfectants, diuretics, enzymes, essential oils, gastro-intestinal agents, haemostatics, lipid regulating agents, local anesthetics, opioid analgesics, parasympathomimetics and anti-dementia drugs, peptides and proteins, sex hormones, stimulating agents, vasodilators or mixtures thereof

Any water insoluble drug may be formulated in the practice of the present invention so as to increase its solubility and hence its bioavailability. Drugs that are particularly useful in the practice of the present invention include but are not limited to aprepitant, bicalutamide, cabergoline, candesartan, celecoxib, cyclosporine, dexamethasone, ezetimibe, fenofibrate, gliclazide, glipizide, griseofulvine, indinavir, isotretinoin, linezolid, modafanil tacrolimus, tamoxifen, telmisartan

The composition of the present invention comprises at least one polymer. The selection of the polymer is very important in the formation of solid solutions. The polymer must increase the dissolution rate and must be pharmacologically in active and non-toxic. Optimum pairing of the drug with the polymer is essential for the formation of a stable solid solution.

Polymers used herein include, but are not limited to water soluble polymers, such as polyethylene glycol (macrogols), polyvinylpyrrolidone, hydroxypropyl methyl cellulose, hydroxyl propyl cellulose, hydroxyethyl cellulose, polyvinyl alcohol, sorbitol, mannitol and saturated polyglycolized glycerides, citric acid, succinic acid

In an embodiment of the present invention, the polymer used for the formation of solid solution is hydroxypropyl methyl cellulose.

The ratio of drug to polymer is in the range of about 1:0.25 to about 1:2.

In one embodiment of the present invention the drug to polymer ratio is about 1:0.5.

Inert pellets used herein include, but are not limited to, microcrystalline cellulose spheres, sugar starch spheres and lactose spheres.

The size of the inert pellets is in the range of about 200 microns to about 1000 microns.

In the present invention solid solutions are formed when the drug and the polymer solution are sprayed onto solid carrier spheres or pellets.

In the present invention, it was surprisingly found that the dissolution of the drug formed as a solid dispersion on the surface of the inert pellet depends on the size of the pellet. It can be seen from Figure I that smaller the particle size of the pellets, faster is the dissolution of the drug in the dissolution medium.

In the present invention, HPMC is used as a water-soluble carrier and several mechanisms are involved which result in the increased dissolution of the drug.

Moreover, it was also found that the amount of residual solvent remaining in the composition is also dependent on the size of the pellet.

One embodiment of the present invention provides a process for the preparation of a stable pharmaceutical composition of aprepitant comprising:
(a) dissolving aprepitant, or a pharmaceutically acceptable salt thereof, and at least one polymer in a suitable solvent, to form a solution;
(b) spraying the solution onto inert pellets; and
(c) drying the inert pellets to remove the solvent.

The present invention relates to a process for the preparation of a solid dispersion of aprepitant and a process for its preparation.

Another embodiment of the present invention provides a solid dispersion of ezetimibe which is prepared as described earlier by spraying the drug-polymer solution onto inert pellets.

A further embodiment of the present invention provides a solid dispersion of glipizide which is prepared as described earlier by spraying the drug-polymer solution onto inert pellets.

The formation of a solid dispersion can be analyzed using thermal analysis, X-ray diffraction, microscopic, spectroscopic or thermodynamic techniques. Figure II represents the XRD pattern of the solid dispersion at the initial stage and after subjecting it to accelerated conditions.

The following examples further illustrate certain specific aspects and embodiments of the invention in detail and are not intended to limit the scope of the invention.

### Examples

### Example 1

| Name of | | | | |
|---|---|---|---|---|
| Ingredient | B1 | B2 | B3 | B4 |
| Aprepitant | 125.000 | 125.000 | 125.000 | 125.000 |
| Hydroxypropyl | 62.500 | 62.500 | 62.500 | x |
| methyl | | | | |
| cellulose(E6) | | | | |
| Hydroxypropyl | x | x | x | 125.000 |
| methyl cellulose | | | | |
| (E15) | | | | |
| Microcrystalline | 150.000 | x | x | x |
| cellulose pellets | | | | |
| (Ethisphere 450) | | | | |
| Microcrystalline | x | 150.000 | x | 12.500 |
| cellulose pellets | | | | |
| (Celphere CP | | | | |
| 507) | | | | |
| Microcrystalline | x | x | 250.000 | x |
| cellulose pellets | | | | |
| (Celphere CP | | | | |
| 305) | | | | |
| Microcrystalline | x | x | x | 400.000 |
| cellulose pellets | | | | |
| (Ethisphere 850) | | | | |
| Sodium lauryl | 0.670 | 0.670 | 0.670 | x |
| sulphate | | | | |
| Propylene glycol | | | | 12.500 |
| Total Fill weight | 338.170 | 338.170 | 438.170 | 675.000 |
| Starting Pellet | 355-500 | 500-710 | 300-500 | 710-1000 |
| size | microns | microns | microns | microns |

Process:
1. Aprepitant was dissolved in a mixture of methanol and dichloromethane.
2. Hydroxypropyl methyl cellulose was dissolved in the same mixture till a clear solution is obtained.
3. Propylene glycol was added to the above mixture and mixed for 10 mins.
4. The above solution was filtered through 100 mesh.
5. The above solution was layered onto the microcrystalline pellets using Fluid bed coater and dried further.
6. Drug loaded pellets were filled into hard gelatin capsules.

### Example 2

### Dissolution Testing of the B1 to B4

Dissolution testing of batches B1 to B4 were performed. The dissolution testing was done using 900 ml water with 2.2% sodium lauryl sulphate as the dissolution medium in USP type II apparatus at 100 rpm. The following results were obtained.

| Time in mins | B1 | B2 | B3 | B4 |
|---|---|---|---|---|
| 0 | 0 | 0 | 0 | 0 |
| 5 | 14 | 12 | 19 | 2 |
| 10 | 34 | 23 | 50 | 14 |
| 15 | 62 | 42 | 77 | 38 |
| 20 | 80 | 54 | 89 | 60 |
| 30 | 97 | 75 | 94 | 94 |
| 45 | 101 | 95 | 96 | 104 |
| 60 | 101 | 100 | 96 | 106 |

### Example 3

### Effect of Pellet Particle Size on Residual Solvent

| Name of Ingredient | B5 | B6 |
|---|---|---|
| Aprepitant | 125.000 | 125.000 |
| Hydroxypropyl methyl | 62.500 | 62.500 |
| cellulose(E6) | | |
| Microcrystalline cellulose | 150.000 | x |
| pellets (Celphere CP 507) | | |
| Microcrystalline cellulose | x | 250.000 |
| pellets (Celphere CP 305) | | |
| Sodium lauryl sulphate | 0.670 | 0.670 |
| Empty hard gelatin capsules | Size ‘1’ | Size ‘0’ |
| Total Fill weight | 338.170 | 438.170 |
| Starting Pellet size | 500-710 | 300-500 |
| | microns | microns |

Process:
1. Aprepitant was dissolved in a mixture of methanol and dichloromethane.
2. Hydroxypropyl methyl cellulose was dissolved in the same mixture till a clear solution is obtained.
3. The above solution was filtered through 100 mesh.
4. The above solution was layered onto the microcrystalline pellets using Fluid bed coater and dried further.
5. Drug loaded pellets were filled into hard gelatin capsules.

Residual Solvent

Residual solvent was measured by gas chromatography using the column DB-624 and carrier gas as helium with a temperature of 40° C. for 7 minutes followed by a rise to 220° C. at the rate of 50° C. per minute and maintaining at 220° C. for 2 minutes. The following results were obtained.

| Name of Solvent | B5 | B6 | Limits |
|---|---|---|---|
| Methanol | 2325 ppm | 640 ppm | NMT 3000 ppm |
| Methylene | 871 ppm | 286 ppm | NMT 600 ppm |
| Chloride | | | |

### Example 4

### Stability of Aprepitant Pellets

Composition

| Name of Ingredient | B7 |
|---|---|
| Aprepitant | 125.000 |
| Hydroxypropyl methyl | 125.000 |
| cellulose | |
| Microcrystalline cellulose | 400.000 |
| pellets (Ethisphere 850) | |
| Propylene Glycol | 12.500 |
| Empty hard gelatin capsules | |
| Total Fill weight | 662.500 |
| Starting Pellet size | 710-1000 |
| | microns |

Process:
1. Aprepitant was dissolved in a mixture of methanol and dichloromethane.
2. Hydroxypropyl methyl cellulose was dissolved in the same mixture till a clear solution is obtained.
3. The above solution was filtered through 100 mesh.
4. The above solution was layered onto the microcrystalline pellets using Fluid bed coater and dried further.
5. Drug loaded pellets were filled into hard gelatin capsules.

Stability of Aprepitant Pellets Upon Storage

Aprepitant pellets were exposed to accelerated storage condition i.e the capsules were stored at about 40° C. and 75% relative humidity. The content of Aprepitant and the total impurity were measured by HPLC immediately after preparing and after 1, 2 and 3 months after accelerated storage condition. HPLC was performed on Xterra RP 18 column using buffer └ortho-phosphoric acid in water┘ and acetonitrile as the mobile phase. The following data was obtained.

| Period | Condition | Total Impurity | Assay in % |
|---|---|---|---|
| Initial | — | 0.25 | 105.5 |
| 1 M | 40° C./75% RH | 0.21 | 102.6 |
| 2 M | 40° C./75% RH | 0.21 | 103.4 |
| 3 M | 40° C./75% RH | 0.21 | 105.0 |

In-Vitro Dissolution Data of Aprepitant Pellets on Storage.

The dissolution testing was done using 900 ml water with 2.2% sodium lauryl sulphate as the dissolution medium in USP type II apparatus at 100 rpm. The following results were obtained

| | | Time [min] | | | | | | |
|---|---|---|---|---|---|---|---|---|
| Period | Condition | 5 | 10 | 15 | 20 | 30 | 45 | 60 |
| Initial | — | 2 | 14 | 38 | 60 | 94 | 104 | 106 |
| 1 M | 40° C./75% RH | 7 | 23 | 38 | 66 | 100 | 110 | 113 |
| 2 M | 40° C./75% RH | 9 | 25 | 40 | 65 | 91 | 107 | 111 |
| 3 M | 40° C./75% RH | 4 | 20 | 40 | 67 | 99 | 107 | 110 |

### Example 5

| Name of Ingredient | Amount |
|---|---|
| Fenofibrate | 150 mg |
| Hydroxypropyl methyl | 250.000 |
| cellulose | |
| Microcrystalline cellulose | 400.000 |
| pellets (Ethisphere 850) | |
| Propylene Glycol | 12.500 |
| Empty hard gelatin capsules | |
| Total Fill weight | 662.500 |
| Starting Pellet size | |

Process
1. Fenofibrate is dissolved in a mixture of methanol and dichloromethane.
2. Hydroxypropyl methyl cellulose is dissolved in the same mixture till a clear solution is obtained.
3. The above solution is filtered through 100 mesh.
4. The above solution is layered onto the microcrystalline pellets using fluid bed coater and dried further.
5. Drug loaded pellets are filled into hard gelatin capsules.

### Example 6

| Name of Ingredient | Amount |
|---|---|
| Ezetimibe | 10 mg |
| Hydroxypropyl methyl | 20.000 |
| cellulose | |
| Microcrystalline cellulose | 400.000 |
| pellets (Ethisphere 850) | |
| Propylene Glycol | 12.500 |
| Empty hard gelatin capsules | |
| Total Fill weight | 662.500 |
| Starting Pellet size | |

Process
1. Ezetimibe is dissolved in a mixture of methanol and dichloromethane.
2. Hydroxypropyl methyl cellulose is dissolved in the same mixture till a clear solution is obtained.
3. The above solution is filtered through 100 mesh.
4. The above solution is layered onto the microcrystalline pellets using Fluid bed coater and dried further.
5. Drug loaded pellets are filled into hard gelatin capsules.

### Example 7

| Name of Ingredient | Amount |
|---|---|
| Telmisartan | 80 mg |
| Hydroxypropyl methyl | 160.000 |
| cellulose | |
| Microcrystalline cellulose | 400.000 |
| pellets (Ethisphere 850) | |
| Propylene Glycol | 12.500 |
| Empty hard gelatin capsules | |
| Total Fill weight | 662.500 |
| Starting Pellet size | |

Process
1. Telmisartan is dissolved in a mixture of methanol and dichloromethane.
2. Hydroxypropyl methyl cellulose is dissolved in the same mixture till a clear solution is obtained.
3. The above solution is filtered through 100 mesh.
4. The above solution is layered onto the microcrystalline pellets using Fluid bed coater and dried further.
5. Drug loaded pellets are filled into hard gelatin capsules.

### Example 8

| Name of Ingredient | Amount |
|---|---|
| Raloxifene | 60 mg |
| Hydroxypropyl methyl | 120 mg |
| cellulose | |
| Microcrystalline cellulose | 400 mg |
| pellets (Ethisphere 850) | |
| Propylene Glycol | 12.5 mg |
| Empty hard gelatin capsules | |
| Total Fill weight | 592.5 mg |
| Starting Pellet size | |

Process
1. Raloxifene HCl is dissolved in a mixture of methanol and dichloromethane.
2. Hydroxypropyl methyl cellulose is dissolved in the same mixture till a clear solution is obtained.
3. The above solution is filtered through 100 mesh.
4. The above solution is layered onto the microcrystalline pellets using Fluid bed coater and dried further.
5. Drug loaded pellets are filled into hard gelatin capsules.

### Example 9

| Name of Ingredient | Amount |
|---|---|
| Glipizide | 5 mg |
| Hydroxypropyl methyl | 10 mg |
| cellulose | |
| Microcrystalline cellulose | 400 mg |
| pellets (Ethisphere 850) | |
| Propylene Glycol | 15 mg |
| Empty hard gelatin capsules | |
| Total Fill weight | 430 mg |
| Starting Pellet size | |

Process
1. Glipizide is dissolved in a mixture of methanol and dichloromethane.
2. Hydroxypropyl methyl cellulose is dissolved in the same mixture till a clear solution is obtained.
3. The above solution is filtered through 100 mesh.
4. The above solution is layered onto the microcrystalline pellets using Fluid bed coater and dried further.
5. Drug loaded pellets are filled into hard gelatin capsules.
   Other drugs and other strengths may be prepared in a similar manner by altering the ratio of drug to HPMC, the fill weight and, if necessary, changing the capsule size.

What is claimed is:

1. A stable pharmaceutical composition comprising a poorly soluble drug, or a pharmaceutically acceptable salt thereof, at least one polymer, and inert pellets, wherein the dissolution rate of said poorly soluble drug is dependent on the particle size of said inert pellets.

2. The pharmaceutical composition of claim 1 wherein said composition is a solid dispersion.

3. The pharmaceutical composition of claim 1 wherein said solid dispersion is formed on said inert pellets.

4. The pharmaceutical composition of claim 1 wherein said polymer is hydroxylpropylmethyl cellulose, polyethylene glycol, polyvinylpyrrolidone, hydroxylpropyl cellulose or hydroxyethyl cellulose.

5. The pharmaceutical composition of claim 4 wherein said polymer is hydroxylpropylmethyl cellulose.

6. The pharmaceutical composition of claim 4 wherein the ratio of said drug to said polymer is in the range of about 1:0.25 to about 1:2.

7. The pharmaceutical composition of claim 1 wherein said inert pellets are microcrystalline cellulose spheres, sugar starch spheres or lactose spheres.

8. The pharmaceutical composition of claim 7 wherein said inert pellets are microcrystalline cellulose spheres.

9. The pharmaceutical composition of claim 1 wherein the particle size of said inert pellets is in the range of about 300 microns to about 1000 microns.

10. The pharmaceutical composition of claim 1 wherein said poorly soluble drug is an antibacterial, antacid, analgesic, anti-inflammatory agent, anti-arrhythmic agent, antiprotozoal agent, anti-coagulant, antidepressant, anti-diabetic agent, antiepileptic agent, antifungal agent, antihistamine, antihypertensive agent, antimuscarnic agent, antineoplastic agent, antimetabolite, antimigraine agent, anti-Parkinsonian agent, antipsychotic, hypnotic agent, sedating agent, antistroke agent, antitussive, antiviral, cardiac inotropic agent, corticosteroid, disinfectant, diuretic, enzyme, essential oil, gastrointestinal agent, haemostatic, lipid regulating agent, local anesthetic, opioid analgesic, parasympathomimetic, antidementia drug, peptide, protein, sex hormone, stimulating agent, or vasodilator.

11. The pharmaceutical composition of claim 1 wherein said poorly soluble drug is aprepitant, bicalutamide, cabergoline, candesartan, celecoxib, cyclosporine, dexamethasone, ezetimibe, fenofibrate, gliclazide, glipizide, griseofulvin, indinavir, isotretinoin, linezolid, modafanil, tacrolimus, tamoxifen, or telmisartan.

12. A stable pharmaceutical composition comprising aprepitant, or pharmaceutically acceptable salt thereof, a polymer and inert pellets, wherein the dissolution rate of aprepitant is dependent on the particle size of the inert pellets.

13. The pharmaceutical composition of claim 12 wherein said composition is a solid dispersion.

14. The pharmaceutical composition of claim 13 wherein said solid dispersion is formed on said inert pellets.

15. The pharmaceutical composition of claim 12 wherein said polymer is hydroxylpropylmethyl cellulose, polyethylene glycol, polyvinylpyrrolidone, hydroxyl propyl cellulose or hydroxyethyl cellulose.

16. The pharmaceutical composition of claim 15 wherein said polymer is hydroxylpropylmethyl cellulose.

17. The pharmaceutical composition of claim 15 wherein the ratio of aprepitant to said polymer is in the range of about 1:0.25 to about 1:2.

18. The pharmaceutical composition of claim 12 wherein said inert pellets are microcrystalline cellulose spheres, sugar starch spheres or lactose spheres.

19. The pharmaceutical composition of claim 18 wherein said inert pellets are microcrystalline cellulose spheres.

20. The pharmaceutical composition of claim 12 wherein the particle size of said inert pellet is in the range of about 300 microns to about 1000 microns.

21. A process for the preparation of a pharmaceutical composition comprising a poorly soluble drug, comprising the steps of:
a. dissolving said drug, or a pharmaceutically acceptable salt thereof, and at least one polymer in a suitable solvent, to form a solution;
b. spraying the solution onto inert pellets; and
c. drying the inert pellets to remove the solvent;
wherein the dissolution rate of said drug is dependent on the particle size of said inert pellets.

22. The process of claim 21 wherein said polymer is hydroxylpropylmethyl cellulose, polyethylene glycol, polyvinylpyrrolidone, hydroxylpropyl cellulose or hydroxyethyl cellulose.

23. The process of claim 22 wherein said polymer is hydroxylpropylmethyl cellulose.

24. The process of claim 21 wherein the ratio of said drug to said polymer is in the range of about 1:0.25 to about 1:2.

25. The process of claim 21 wherein said inert pellets are microcrystalline cellulose spheres, sugar starch spheres or lactose spheres.

26. The process of claim 25 wherein said inert pellets are microcrystalline cellulose spheres.

27. The process of claim 21 wherein said drug is an antibacterial, antacid, analgesic, anti-inflammatory agent, anti-arrhythmic agent, antiprotozoal agent, anti-coagulant, antidepressant, anti-diabetic agent, antiepileptic agent, antifungal agent, antihistamine, antihypertensive agent, antimuscarnic agent, antineoplastic agent, antimetabolite, antimigraine agent, anti-Parkinsonian agent, antipsychotic, hypnotic agent, sedating agent, antistroke agent, antitussive, antiviral, cardiac inotropic agent, corticosteroid, disinfectant, diuretic, enzyme, essential oil, gastrointestinal agent, haemostatic, lipid regulating agent, local anesthetic, opioid analgesic, parasympathomimetic, antidementia drug, peptide, protein, sex hormone, stimulating agent, or vasodilator.

28. The process of claim 21 wherein said drug is aprepitant, bicalutamide, cabergoline, candesartan, celecoxib, cyclosporine, dexamethasone, ezetimibe, fenofibrate, gliclazide, glipizide, griseofulvin, indinavir, isotretinoin, linezolid, modafanil, tacrolimus, tamoxifen, or telmisartan.

29. A process for the preparation of a pharmaceutical composition comprising aprepitant, or a pharmaceutically acceptable salt thereof, comprising:
a. dissolving aprepitant, or a pharmaceutically acceptable salt thereof, and at least one polymer in a suitable solvent, to form a solution;
b. spraying said solution onto inert pellets; and
c. drying said inert pellets to remove the solvent;
wherein the dissolution rate of aprepitant is dependent on the particle size of said inert pellets.

30. The process of claim 29 wherein said polymer is hydroxylpropylmethyl cellulose, polyethylene glycol, polyvinylpyrrolidone, hydroxylpropyl cellulose or hydroxyethyl cellulose.

31. The process of claim 30 wherein said polymer is hydroxypropylmethyl cellulose.

32. The process of claim 29 wherein the concentration of said hydroxypropylmethyl cellulose is in the range of about 7.5% to about 20%.

33. The process of claim 29 wherein the ratio of aprepitant to hydroxypropylmethyl cellulose is in the range of about 1:0.25 to about 1:2.

34. The process of claim 29 wherein said inert pellets are microcrystalline cellulose spheres, sugar starch spheres or lactose spheres.

35. The process of claim 34 wherein said inert pellets are microcrystalline cellulose spheres.
